(19) United States
(12) Patent Application Publication
(10) Pub. No.: US 2010/0008988 A1
(43) Pub. Date: Jan. 14, 2010

(54) TABLET COMPOSITIONS OF AMINE POLYMERS

(75) Inventors: KAMAL MEHTA, RAJASTHAN (IN); SRINIVAS GANGA ARRA, NAVI MUMBAI- 400709 (IN); HIDAYTULLA AGA, SATARA (IN)

Correspondence Address: GLENMARK PHARMACEUTICALS INC USA, 750 CORPORATE DRIVE, MAHWAH, NJ, 07430, US

(73) Assignee: GLENMARK GENERICS, LTD., MUMBAI-400 (IN)

(21) Appl. No.: 12/501,620

(22) Filed: Jul. 13, 2009

Related U.S. Application Data
(60) Provisional application No. 61/177,672, filed on May 13, 2009.

(30) Foreign Application Priority Data
Jul. 14, 2008 (IN) 1475/MUM/2009

(51) Int. Cl. A61K 9/28; A61K 31/765; A61P 7/00

(52) U.S. Cl. 424/474; 424/78.38

(57) ABSTRACT

The present invention provides pharmaceutical compositions, essentially comprising less than about 95% by weight of an aliphatic amine polymer. The present invention relates to pharmaceutical compositions comprising aliphatic amine polymers of sevelamer hydrochloride, sevelamer carbonate and colesevelam hydrochloride; and methods of preparing pharmaceutical compositions thereof.

### PRIORITY

This application claims the benefit to Indian Provisional Application 1475/MUM/2008, filed on Jul. 14, 2008, under 35 U.S.C. §119 to U.S. Provisional Application 61/177,672, filed on May 13, 2009, the contents of each of which are incorporated by reference herein in their entirety.

### BACKGROUND OF THE INVENTION

1. Technical Field

The present invention relates to pharmaceutical compositions comprising aliphatic amine polymers and methods of their preparation.

2. Description of the Related Art

Phosphate-binding polymers, which are non-absorbed polymers capable of binding phosphate, are useful as remedies for hyperphosphatemia induced by renal hypofunction such as renal insufficiency. For instance, U.S. Pat. No. 5,496,545 discloses phosphate-binding polymers, which are cationic polymer compounds, comprising primary and secondary amines which are prepared by crosslinking polyallyamine with the use of a crosslinking agent such as epichlorhydrin.

A variety of aliphatic amine polymers have been found to be useful as phosphate binders. In particular, U.S. Pat. Nos. 5,496,545 and 5,667,775 disclose aliphatic amine polymers which are reported to bind phosphate from patients suffering from renal failure.

Alkylated aliphatic amine polymers, which are disclosed in U.S. Pat. Nos. 5,624,963 and 5,679,717 and in Patent Publications WO98/29107 and WO99/22721, are useful cholesterol lowering agents.

A pharmaceutical composition containing an aliphatic amine polymer is described in U.S. Pat. No. 6,733,780 (the '780 patent). The '780 patent discloses a tablet core, which comprises at least about 95% by weight of an aliphatic amine polymer. The '780 patent also discloses a method of producing a tablet core comprising at least about 95% by weight of an aliphatic amine polymer; comprising (1) hydrating the aliphatic amine polymer to the desired moisture level; (2) blending the aliphatic amine polymer with excipients in amounts such that the polymer comprises at least about 95% by weight of the resulting blend; and (3) compressing the blend to form a tablet core.

Compositions comprising aliphatic amine polymers such as for example sevelamer hydrochloride as the active pharmaceutical ingredient is described in U.S. Patent Publication 2007/0190020, wherein the aliphatic amine polymers are spray granulated.

U.S. Pat. No. 6,383,518 describes a tablet comprising a phosphate-binding polymer of an average particle size of 400μ or less, and crystalline cellulose and/or low substituted hydroxypropylcellulose.

### SUMMARY OF THE INVENTION

The present invention provides pharmaceutical composition, comprising less than about 95% by weight of an aliphatic amine polymer.

The present invention relates to the pharmaceutical composition, comprising less than about 95% by weight of an aliphatic amine polymer, wherein an aliphatic amine polymer is selected from sevelamer hydrochloride, sevelamer carbonate and colesevelam hydrochloride.

The present invention provides pharmaceutical tablet composition, comprising less than about 95% by weight of an aliphatic amine polymer.

The present invention provides pharmaceutical tablet composition comprising less than about 80% by weight of an aliphatic amine polymer.

The present invention provides pharmaceutical tablet composition comprising less than about 70% by weight of an aliphatic amine polymer.

The present invention further provides pharmaceutical tablet composition, comprising, a core having less than about 95% by weight of an aliphatic amine polymer selected from the group consisting of sevelamer hydrochloride, sevelamer carbonate and colesevelam hydrochloride, and a coat.

The present invention further provides pharmaceutical tablet composition comprising, a core having less than about 80% by weight of an aliphatic amine polymer selected from the group consisting of sevelamer hydrochloride, sevelamer carbonate and colesevelam hydrochloride, and a coat.

The present invention further provides pharmaceutical tablet composition comprising, a core containing less than about 70% by weight of an aliphatic amine polymer selected from the group consisting of sevelamer hydrochloride, sevelamer carbonate and colesevelam hydrochloride, and a coat.

In one of the aspects of the present invention, it is preferred that, the core of the tablet of present invention, does not consists of additives like crystalline cellulose and/or low substituted hydroxypropylcellulose, if the aliphatic amine polymer present therein is sevelamer hydrochloride, sevelamer carbonate and colesevelam hydrochloride.

The present invention further provides a method of preparing a pharmaceutical tablet composition comprising less than about 95% by weight of an aliphatic amine polymer.

### DETAILED DESCRIPTION OF THE INVENTION

The present invention relates to pharmaceutical compositions comprising an aliphatic amine polymer, selected from the group comprising of sevelamer hydrochloride, sevelamer carbonate and colesevelam hydrochloride, and to the methods of preparation thereof. More particularly, the present invention provides pharmaceutical composition comprising a tablet core comprising less than about 95%, preferably less than 80%, more preferably less than 70% by weight of an aliphatic amine polymer.

The present invention provides a a pharmaceutical composition in the form of a tablet, comprising a core containing an aliphatic amine polymer plus one or more pharmaceutically acceptable excipients, and a film coat upon the core tablet.

The aliphatic amine polymer resin can be any of the aliphatic amine resins described in U.S. Pat. Nos. 5,496,545; 5,667,775; 5,703,188; 5,679,717; 5,693,675, 5,607,669; and 5,618,530, each of which is hereby incorporated herein by reference in its entirety. Preferably, the aliphatic amine polymer is selected from sevelamer hydrochloride (HCl), sevelamer carbonate and colesevelam hydrochloride.

The present invention provides that the tablet further comprises, fillers, glidants, lubricants and binders, not limited to the sucrose, mannitol, microcrystalline cellulose, lactose monohydrate, colloidal silicon dioxide, stearic acid, magnesium silicate, calcium silicate calcium stearate, glyceryl behenate, magnesium stearate, talc, zinc stearate, sodium stearylfumarate, hydroxypropylmethylcellulose (HPMC) and polyvinyl pyrrolidone.

The present invention provides that the tablet, comprises upto about 50%, preferably upto about 30%, of pharmaceutically acceptable excipients, by the total weight of tablet.

The film coating comprises a film forming polymer and a plasticizer. A film forming polymer can be selected from but not limited cellulosic ethers such as hydroxypropylmethylcellulose (HPMC) and hydroxypropyl cellulose. The plasticizer can be, for example, an acetylated monoglyceride such as diacetylated monoglyceride, triacetin, polyethylene glycol, triethyl citrate, a polysorbate, preferably diacetylated monoglyceride. The coating composition can further include a pigment to provide a tablet coating of the desired color. For example, to produce a white coating, a white pigment can be selected, such as titanium dioxide.

The present invention provides an aliphatic amine polymer further comprising a moisture content upto about 10%, preferably upto about 5%, more preferably upto about 2% of the weight of aliphatic amine polymer. The moisture content of aliphatic amine polymer, means the water of hydration, which is water added to wet the aliphatic amine polymer to make it suitable for compression.

For the purpose of present invention, the quantity of aliphatic amine polymer to be incorporated in the tablet, is determined based on the moisture content present in the aliphatic amine polymer.

The present invention provides a method of producing a tablet core, comprising a) uniformly mixing one or more excipients with aliphatic amine polymer, such that the resultant blend consists of less than about 70% aliphatic amine polymer and b) directly compressing the blend into tablets.

The examples are not intended to be limiting of the scope of the present invention but read in conjunction with the detailed and general description above, to provide further understanding of the present invention and an outline of processes for preparing the compositions of the invention.

### EXAMPLES

### Example 1

### Sevelamer Hydrochloride Tablets 800 mg

| S. No. | Excipient | Specs. | mg per tablet |
|---|---|---|---|
| | Core | | |
| 1. | Sevelamer Hydrochloride (5% moisture | IH | 840.00 |
| | content) | | |
| 2. | Lactose Monohydrate | NF | 250.00 |
| 3. | Colloidal Silicon Dioxide | NF | 10.00 |
| 4. | Stearic Acid | NF | 10.00 |
| Net Weight | | | 1070.00 |
| | Coating | | |
| 1. | Hypromellose ® 2910 (HPMC E50) | USP | 28.00 |
| 2. | Hypromellose ® 2910 (HPMC E5) | USP | 28.00 |
| 3. | Diacetylated Monoglyceride | NF | 14.00 |
| 4. | Water | — | q.s. |

| | |
|---|---|
| Net Weight | 1140.00 |

### Manufacturing Procedure

1. Sevelamer hydrochloride and lactose monohydrate were sifted through #40 sieve and mixed for 10 minutes. Colloidal silicon dioxide and stearic acid were sifted through #40 sieve, and then added to the above blend and mixed for another 5 minutes.
2. The above blend was then compressed into tablets using suitable tools.
3. The coating solution was prepared by dissolving acetylated monoglyceride in purified water under stirring followed by addition of HPMC E50LV® into it, under stirring until uniform dispersion formed.
4. The coating solution of 3) was sprayed onto the core tablets of 2) using a suitable coating machine to achieve a weight gain between 6-8% w/w per tablet.

### Example 2

### Sevelamer Hydrochloride Tablets 800 mg

| S. No. | Excipient | Specs. | mg per tablet |
|---|---|---|---|
| | Core | | |
| 1. | Sevelamer Hydrochloride (8% moisture | IH | 864.00 |
| | content) | | |
| 2. | Lactose Monohydrate | NF | 340.00 |
| 3. | Colloidal Silicon Dioxide | NF | 10.00 |
| 4. | Stearic Acid | NF | 10.00 |
| Net Weight | | | 1224.00 |
| | Coating | | |
| 1. | Hypromellose ® 2910 (HPMC E 50) | USP | 28.00 |
| 2. | Hypromellose ® 2910 (HPMC E 5) | USP | 28.00 |
| 3. | Diacetylated Monoglyceride | NF | 14.00 |
| 4. | Water | — | q.s. |

| | |
|---|---|
| Net Weight | 1294.00 |

### Manufacturing Procedure

1. Sevelamer hydrochloride and lactose monohydrate were sifted through 40# sieve and mixed for 10 minutes. Colloidal silicon dioxide and stearic acid were sifted through 40# sieve, and added to the above blend and mixed for another 5 minutes.
2. The above blend was then compressed into tablets using suitable tools.
3. The coating solution was prepared by dissolving acetylated monoglyceride in purified water under stirring followed addition of HPMC E50LV® into it, under stirring until uniform dispersion formed.
4. The coating solution of 3) was sprayed onto the core tablets of 2) using a suitable coating machine to achieve a weight gain between 6-8% w/w per tablet.

### Example 3

### Colesevelam Hydrochloride Tablets 625 mg

| Ingredients | Specs | Mg/tablet |
|---|---|---|
| Intra granular | | |
| Colesevelam Hydrochloride anhydrous | USP | 625.00 |
| Microcrystalline cellulose (Avicel ® PH 101) | USP | 220.00 |
| Hydroxypropyl methyl cellulose (HPMC E5LV ®) | USP | 20.00 |
| Colloidal Silicon dioxide | USP | 15.0 |
| Hydroxypropyl methyl cellulose (K4M ®) | USP | 100.0 |
| Lubrication | — | — |
| Magnesium Stearate | USP | 10.0 |
| Core Tablet weight (mg) | — | 990 |
| Coating (PART B) | | |
| Hydroxypropyl methyl cellulose (HPMC E50LV ®) | USP | 69.80 |
| Diacetylated monoglyceride | USP | 9.40 |
| P. Water | | q.s |
| Coated Tablet weight (mg) | | 1069.2 |

### Manufacturing Process

1. Colesevelam Hydrochloride, Microcrystalline cellulose, HPMC ES LV®, Hydroxypropyl methyl cellulose (K4M®) and Aerosil®-200 were sifted through 40# mesh and mixed for 10 minutes in bin blender. Magnesium stearate, sifted through #60 mesh and added to the above blend and mixed for another 5 min.
2. The above lubricated blend is then compressed into tablets
   Coating of Tablets p0 3. The coating solution was prepared by dissolving acetylated monoglyceride in purified water under stirring followed addition of HPMC E50LV® into it, under stirring until uniform dispersion formed.
4. The coating solution of 3) is sprayed onto the core tablets of 2) using a suitable coating machine to achieve a weight gain between 6-8% w/w per tablet.

### Example No 4

### Colesevelam Hydrochloride Tablets

| Ingredients | Function | Mg/tablet |
|---|---|---|
| Wet Granulation (PART A) | | |
| Intra granular | | |
| Colesevelam Hydrochloride | Active | 625.00 |
| Microcrystalline cellulose (Avicel PH 101) | Diluent | 220.00 |
| Hydroxy propyl methyl cellulose | Binder | 20.00 |
| (HPMC E5LV) | | |
| Silicon Dioxide (Aerosil-200) | Glidant | 25.00 |
| Purified water | Granulating | Qs. |
| | fluid | |
| Extragranular | — | — |
| Hydroxy propyl methyl cellulose | Binder | 20.00 |
| (HPMC K4M) | | |
| Magnesium Stearate | Lubricant | 10.00 |
| Core Tablet weight (mg) | — | 920 |
| Coating (PART B) | | |
| Hydroxy propyl methyl cellulose | Film former | 80.60 |
| (HPMC E50LV) | | |
| Diacetylated monoglyceride | Plasticizer | 9.40 |
| P. water | Solvent | Qs. |
| Coated Tablet weight (mg) | — | 1040.0 |

### Manufacturing Process

1. Colesevelam hydrochloride, microcrystalline cellulose, Aerosil® and half of the quantity of HPMC ES LV® were sifted through 40# mesh mixed for 10 minutes in a rapid mixer granulator (RMG).
2. The half quantity of HPMC ES LV® dissolved in water under stirring.
3. The material of 1) above is granulated with the binder solution of 2 in RMG.
4. The granules of 3 were then dried to achieve loss of drying (LOD) 7-8% in Fluid Bed Dryer and then loaded into a bin blender
5. HPMC K4M® was sifted through #40 mesh and mixed with the granules of 4 for 5 min.
6. Magnesium stearate was sifted through #60, and mixed with above blend of 5 in a bin blender for 5 min.
7. The above lubricated blend is then compressed to tablets.

### Coating of Tablets:

8. The coating solution was prepared by dissolving acetylated monoglyceride in purified water under stirring followed addition of HPMC E50LV® into it, under stirring until uniform dispersion formed.
9. The coating solution of 8) is sprayed onto the core tablets of 7) using a suitable coating machine to achieve a weight gain between 6-8% w/w per tablet

1. A core tablet comprising less than about 95% by weight of an aliphatic amine polymer, wherein the aliphatic amine polymer is selected from sevelamer hydrochloride, sevelamer carbonate and colesevelam hydrochloride.

2. The core tablet of claim 1, wherein the aliphatic amine polymer is less than about 80% by weight of core tablet.

3. The core tablet of claim 1 further comprising at least one filler.

4. The core tablet of claim 3 wherein the filler is selected from microcrystalline cellulose, lactose monohydrate and mannitol.

5. A core tablet comprising (i) less than 95% by weight of aliphatic amine polymer, selected from sevelamer hydrochloride, sevelamer carbonate and colesevelam hydrochloride (ii) microcrystalline cellulose (iii), hydroxypropylmethyl cellulose, and (iv) magnesium stearate.

6. The core tablet of claim 5, comprising (i) the aliphatic amine polymer, less than about 80%, (ii) microcrystalline cellulose from about 15% to about 30% (iii) hydroxypropylmethyl cellulose from about 5% to about 20% by weight, and (iv) magnesium stearate from about 0.5% to about 1.5%, by weight of the core tablet

7. The core tablet of claim 5, wherein the aliphatic amine polymer is sevelamer or its salt.

8. The core tablet of claim 5, wherein the aliphatic amine polymer is colesevelam hydrochloride.

9. The core tablet of claim 5, further coated with a film coat.

10. A process for the preparation of a core tablet, comprising less than about 80% by weight of an aliphatic amine polymer, comprising: (a) blending the polymer with excipients (b) granulating the blend with aqueous or hydro-alcoholic or non-aqueous solvents, and (c) then compressing the granulated blend into tablets; and (d) coating to the tablet obtained in c.

11. The process of claim 10 wherein the aliphatic amine polymer comprises water up to about 10%.

12. The process of claim 10 wherein the aliphatic amine polymer is sevelamer or its salt.

13. The process of claim 10 wherein the aliphatic amine polymer is colesevelam hydrochloride.
